(19) United States
(12) Patent Application Publication
(10) Pub. No.: US 2025/0129396 A1
(43) Pub. Date: Apr. 24, 2025

(54) METHOD OF MAKING A MICROBIAL FATTY ACID AND FATTY ACID BUTYL ESTER USING A HYBRID BIOSOLVENT WASHING/EXTRACTION PROCESS AND MICROBIAL LIPID OIL AND HYDROPHOBIC BIOHYDROCARBON AS A LIQUID FUEL INTERMEDIARY

(71) Applicant: Comstock IP Holdings LLC, Milton, GA (US)

(72) Inventors: David WINSNESS, Alpharetta, GA (US); Michael J. RIEBEL, Mankato, MN (US)

(21) Appl. No.: 18/921,439

(22) Filed: Oct. 21, 2024

Related U.S. Application Data
(60) Provisional application No. 63/591,817, filed on Oct. 20, 2023.

(51) Int. Cl. C12P 7/649; C10L 1/19; C12N 1/06

(52) U.S. Cl. CPC C12P 7/649; C10L 1/19; C12N 1/063

(57) ABSTRACT

A self-generated biosolvent from cellulosic biorefining is used to wash/extract and optionally esterify free fatty acids from microbial oil. Free fatty acids or fatty acid esters and a hydrophobic biohydrocarbon are processed to provide a hybrid liquid fuel intermediary whereas the lipid oil is derived from cellulosic sugars and from microbial conversion of cellulose, hemicellulose, and/cellulosic sugar to lipids. In addition, higher utilization of woody biomass for the production of cellulosic sugars can be further converted to lipid oils, and the remaining hemicellulose/lignin fraction is converted to a novel hydrophobic biohydrocarbon and self-generated biosolvent in which both the processed lipid oils and biohydrocarbon can be reacted together to form a new fuel intermediary. The self-generated biosolvent comprising butanol, butanol esters, and additional biochemicals, can penetrate a microbial cell wall structure to provide higher extraction yields. The process integrates supercritical esterification of the free fatty acids using the self-generated biosolvent blend. Esterification of free fatty acids from microbial oil can be blended with a liquid biohydrocarbon to create a fuel intermediary. A phase separation process for separation of aqueous and hydrophilic materials from hydrophobic liquids is provided, wherein hydrophobic materials are the biosolvent and fatty acid esters.

### CROSS-REFERENCE TO RELATED APPLICATIONS/INCORPORATION BY REFERENCE STATEMENT

This application claims benefit under 35 USC § 119(e) of U.S. Provisional Application No. 63/591,817, filed Oct. 20, 2023, in accordance with the provisions of 35 USC § 21 and 37 CFR § 1.7 (given that Oct. 20, 2024 fell on a Sunday). The entire contents of the above-referenced patent application(s) are hereby expressly incorporated herein by reference.

### BACKGROUND

With an ever-increasing demand for renewable alternatives to conventional fuels, biodiesel has attracted considerable interest in recent years as an environment-friendly energy resource. Biodiesel is primarily obtained by the transesterification of triacylglycerols (TAGs) that make up plant oils (vegetable/seed oils) and animal fats, typically in the presence of a catalyst, yielding fatty acid methyl esters (FAMEs).

More recently these vegetable oil streams are being used as a feedstock for the production of renewable diesel fuels. Thus, there is a need for additional renewable oil feedstocks and renewable carbon fuel intermediaries for the production of biodiesel, renewable diesel, aviation fuels, and conversion into additional drop in liquid fuels.

Converting microbial lipids and hydrophobic biohydrocarbons to fuels is a promising approach to displace conventional fossil fuels, especially for diesel and jet fuels that share the same carbon number range as the microbial lipids. While previous research has focused primarily on algae lipids, the advent of third generation cellulosic sugars may spur further development of heterotrophic oleaginous microbes as well.

Lipids from oleaginous yeasts emerged as a sustainable alternative to vegetable oils and animal fat to produce biodiesel, the biodegradable and environmentally friendly counterpart of petroleum-based diesel fuel. Oleaginous yeasts use sugar as a feedstock that converts the sugar into lipids or oil within the microbe, that can later be extracted.

The economical production of biodiesel, renewable diesel, jet fue,l or other fuels from lignocellulose requires an effective co-fermentation of lignocellulose-derived sugars, such as glucose. This has been problematic to convert woody biomass to sugar using hydrolysis processes given the lignin's ability to inhibit enzymatic processes creating low yields.

Recently, chemical and fuel production from lignocellulosic biomass has received increased attention. Hydrolysates of such biomasses contain mixtures of sugars, mainly glucose and xylose in various ratios. Complete conversion of sugars in hydrolysates is necessary for efficient utilization of lignocellulosic biomass and conversion of cellulosic sugar to liquid fuels. It would be environmentally and economically advantageous if biosolvent blends from lignocellulose treatment could be used to further increase lipid recovery using oleaginous yeasts.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 illustrates one non-limiting embodiment of a general process disclosed herein.

FIG. 2 illustrates one non-limiting embodiment of an “in situ” esterification of microbes into fatty acid esters as disclosed herein.

### DETAILED DESCRIPTION

Before explaining at least one embodiment of the present disclosure in detail by way of exemplary language and results, it is to be understood that the disclosure is not limited in its application to the details of construction and the arrangement of the components set forth in the following description. The disclosed concept(s) is capable of other embodiments or of being practiced or carried out in various ways. As such, the language used herein is intended to be given the broadest possible scope and meaning; and the embodiments are meant to be exemplary and not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, and/or methods disclosed herein can be made and executed without undue experimentation in light of the present disclosure. While the disclosed compositions, assemblies, systems, and methods have been described in terms of particular embodiments, it will be apparent to those of skill in the art that variations may be applied to the compositions and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined herein and in the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open- ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

Certain non-limiting embodiments of the present disclosure relate to methods of processing microbial lipids to free fatty acids integrating self-generated biosolvent blends from cellulosic refining to wash, extract, and optionally esterify free fatty acids.

Certain non-limiting embodiments of the present disclosure also relate to the integration of a hydrophobic biohydrocarbon and processed microbial based free fatty acid or fatty acid butyl esters as to create a blend stock or fuel intermediary for the production of biodiesel, renewable diesel, gasoline, and aviation fuels.

The inventors of this disclosure have previously developed methods of processing woody biomass to sugar with high efficiencies and yields of sugar. For example, U.S. application Ser. No. 16/119,030 (U.S. Publication No. 2019/0062508) titled Method for Separating and Recovering Lignin and Meltable Flowable Biolignin Polymers, filed by Mssrs. Winsness, Riebel and Riebel and U.S. Provisional Application Ser. No. 63/367,874 titled Lignocellulosic Biomass Derived Biointermediates and Renewable Fuels, disclose using a blend of hydrophobic and hydrophilic solvents in addition to fractionations and separation of specific components within the woody biomass leaving an “inhibitor free” cellulosic pulp product that can be converted to sugar using standard enzymatic hydrolysis processes. In addition, this process also converts a good portion of hemicellulose to the self-generated biochemicals and depolymerizes/fractionates the lignin into hydrophobic and hydrophilic portions that are removed from the pulp. This also provides for esterification of the depolymerized lignin (phenols) and the wood extractives found in woody biomass that provides for esterification of the resulting hydrophobic biohydrocarbon liquid that comprises hydrophobic phenols, hydrophobic furans, hydrophobic butanol derivatives, and hydrophobic esters.

The process of converting woody biomass into a pulp that can be further converted to sugar and the co-product of a hydrophobic biohydrocarbon also produces and recycles a hydrophobic solvent mixture typically comprising butyl esters, butyl acetate, butanol, furans, aldehydes, and other self-generated solvents.

This disclosure utilizes this unique self-generated biosolvent blend derived from cellulosic sugar production process for the washing and extraction of oil from microbes in which the microbial oil can be modified and blended with the liquid hydrophobic biohydrocarbon as to form a fuel intermediary.

US Patent Publ. No. 2015/0232497 titled “Method for esterifying lignin with at least one fatty acid” discloses blending an esterify lignin with a fatty acid using acetic anhydride and a catalyst wherein the esterified lignin is then blended with a fatty acid. This also discloses that the fatty acid is a tall oil fatty acid derived from chemical pulp/paper mill operations. The process also can be carried out in the presence of a solvent including toluene, hexane, pyridine, and combinations thereof. In addition, the process includes a solvent selected from the group consisting of acetone, pentane, hexane, heptane, methanol, ethanol, propanol, butanol, pentanol, toluene, any water mixture thereof, and any combination thereof. The lignin to be used in the method is selected from a group consisting of kraft lignin, biomass originating lignin, lignin from alkaline pulping process, lignin from soda process, lignin from organosolv pulping and any combination thereof.

U.S. Pat. No. 9,920,201 to RenFuel, entitled “Compositions of biomass materials for refining” discloses a composition comprising: a) a lignin, b) a solvent, c) a carrier fluid, and d) a fatty acid as to create a fuel intermediary or various additional applications or product usage.

U.S. Pat. No. 5,808,130, entitled “Esterification of Phenols” to Henkel Corporation, 1996, discloses using an acetic anhydride and other acids or anhydride for the esterification of phenols for various coatings and adhesive applications.

U.S. Patent Publ. No. 2016/0355535, entitled “Fatty acid derivatives of lignin and uses thereof” to North Carolina State University, discloses a fatty acid derivative of lignin consisting of a modified lignin and fatty acid, whereas fatty acid derivative is a fatty acid ester. The application of this art generally relates to a thermoplastic polymer application. Within this art they mention the usage of a “polar protic” solvent that includes acetic acid, ethanol, formic acid, isopropanol, methanol, n-butanol, or water.

U.S. Pat. No. 6,172,204, entitled “Compositions based on lignin derivatives,” University of Minnesota, Sarkanen, discloses compositions of a lignin derivative and acylating agents being acid anhydrides, acyl halides and combinations thereof used to create a bioplastic.

U.S. Patent Publ. No. US2017/0044328 to Renmatix, Inc., entitled “Upgrading lignin from lignin-containing residues through reactive extraction,” discloses methods of functionalizing lignin.

U.S. Patent Publ. No. 2019/0010419 discloses a process for extracting lipids for use in production of biofuels (DSM), discloses removing oil from microbes using a solvent wherein the solvent may include hexane, dodecane, decane, diesel, one or more alcohols, or combinations thereof.

U.S. Patent Publ. No. 2014/0373432 to Shell Oil titled “Direct method of producing fatty acid esters from microbial biomass,” discloses producing a fatty acid ester from a microbial biomass using a mineral acid and alcohol wherein the alcohol can comprise at least one alcohol selected from methanol, ethanol, propanol, butanol, hexanol, heptanol, octanol, nonanol, or decanol. Methanol is their preferred for producing fatty acid methyl ester (FAME).

Microbial lipids are viewed as an alternative feedstock for biofuel production because fatty acid compositions of accumulated lipids are similar to vegetable oils currently used as feedstock for the production of first-generation biodiesel and renewable diesel fuels. Microbial lipids are also known as single cell oils (SCO), and are produced by a heterogeneous group of oleaginous microorganisms that include less than a hundred species of different microbial species including yeasts, fungi, bacteria, and algae.

The bioconversion of lignocellulose to the microbial lipids includes following steps: pretreatment of lignocellulose biomass, hydrolysis of structural carbohydrates to fermentable sugars, microbial production of lipids, and isolation and purification of the product. Since most of the oleaginous microorganisms lack cellulose and hemicellulose activity, structural polysaccharides in lignocellulosic biomass must be hydrolyzed to fermentable sugars (mainly xylose and glucose), which microorganisms can use as a carbon source.

The organic layer from a novel biorefining process comprising hydrophobic depolymerized phenols, hydrophobic esters, hydrophobic furans, butyl esters, and butanol represents the organic layer within the process disclosed. U.S. Pat. No. 9,365,525 to American Science and Technology discloses a method to create a butanol hydrophobic hybrid solvent from woody biomass that is self-generated as to remove lignin from pulp. In U.S. Patent Publ. No. 2019/0062508, Winsness and Riebel provide for the addition of carrier fluids to the butanol based organic layer prior to recovery of the butanol and related self-generated biosolvents and depolymerized lignin fractionation of woody biomass into a hydrophobic melt-flowable lignin and pulp. U.S. Provisional Pat. Appl. 63/367,874 provides a method of creating an enzyme inhibitor free pulp for sugar conversion, a purified organic liquid comprising hydrophobic phenols, hydrophobic esters, hydrophobic furans in which the process has the ability to blend reactive functional additives, washing steps and modifications to the organic layer and resulting biohydrocarbon material or liquid. These three patents are each incorporated herein by reference thereto in their entirety.

This unique organic layer liquid comprises both a biohydrocarbon and excess of a self-generated biosolvent in which the biosolvent fraction is used in this disclosure to wash, extract, and optionally esterify lipid oils.

The disclosure also improves the yield and total output of future cellulosic biorefineries wherein cellulose is first converted to sugars. The sugar can then be processed into a lipid oil using oleaginous microorganisms. The oil containing microorganisms are then processed using various methods to “lyse” or crack the microbe. The hydrophobic organic layer liquid or hydrophobic self-generated recovery biosolvent from the biorefinery can then be used to “wash and extract” the microbial oils providing for a hybrid liquid fuel intermediary.

The disclosure herein also provides for an optional esterification of the free fatty acids that have been extracted using the hydrophobic blend of self-generated biosolvents or hydrophobic blend of self-generated biosolvents with depolymerized lignin esters and other esters in which the esterified free fatty acids and esterified organic layer are compatible and provide for a hydrophobic hybrid fuel intermediary blend for further hydroprocessing into a “drop in” fuel.

The disclosure also provides for a more efficient method for the washing and extraction of microbial lipids based on a hybrid blend of biosolvents produced from cellulosic fuel process wherein the biosolvent or components within the biosolvent have the ability to penetrate the microbial cell was structure.

In one embodiment the hydrophobic biosolvents (“HBS”), such as butyl acetate, butyl ester, oleoresins, organic furans, fatty acids, rosins, terpenes, wood extracts, hemicellulose derivatives, such as furfural and 5-hydroxymethylfurfural (“HMF”).

As described in detail hereinafter, a self-generated biosolvent comprises butanol, butanol esters, and additional biochemicals that can penetrate a microbial cell wall structure to provide higher oil extraction yields from the microbes. Supercritical esterification of the free fatty acids can be integrated using a self-generated biosolvent blend. Esterification of free fatty acids from microbial oil can be blended with a liquid biohydrocarbon as to create a fuel intermediary. Phase separation processes for separation of aqueous and hydrophilic materials from hydrophobic liquids are described wherein hydrophobic materials comprise the biosolvent and fatty acid esters.

### Sugar to Lipids

Single cell oils (SCOs) are intracellular storage lipids comprising triacylglycerols (TAGs). SCOs are produced by oleaginous microorganisms that are able to accumulate between 20% and up to 80% lipid per dry biomass in the stationary growth phase under nutrient limitations, e.g., nitrogen or phosphor, with simultaneous excess of carbon source. Depending on the oleaginous microorganism including bacterial, yeast, microalgae, or fungal species, the fatty acid profile of SCOs can vary, making them highly suitable for diverse industrial applications.

Considering the foreseeable depletion of crude oil, the highly controversial “food-or-fuel” discussion about using plant oils for biodiesel production, the usage of non-food woody biomass has been explored for the production of various fuels and sugars derived from the hydrolysis of cellulose into sugars.

“Third-generation” biodiesel is generally obtained from oil-producing microorganisms, for example, microalgae, bacteria, and yeasts. Single cell oils (SCO) are produced by oleaginous unicellular microorganisms by utilizing various waste materials as sources of carbon and nutrition. SCOs have been lately considered as subjects for active research, essentially in the light of increasing cost of oil.

Some yeasts accumulate lipids to over 20% of the cell dry weight and are thus termed oleaginous yeasts. The carbon-chain lengths of the accumulated fatty acids typically range from 12 to 24, with the major components being palmitic acid (C16:0), stearic acid (C18:0), oleic acid (C18:1), and linoleic acid (C18:2). The composition is similar to that of plant oils; therefore, these lipids can be used as feedstocks for biofuels and oleochemical products.

Oleaginous yeast, which has an inherent ability to accumulate lipids from 20% to 70% as a percentage of cell dry weight, offers many advantages to overcome challenges associated with lignocellulose-based lipid production. Basidiomycetous yeast species such as Cryptococcus albidus and Trichosporon oleaginosus are known to enable use of a variety of carbon sources, and can be grown without supplemented costly nutrients. In addition, oleaginous yeast cultures are insusceptible to toxic compounds compared with bacteria.

Some bacteria from the actinomycetes group, such a Nocardia, Rhodococcus, and Mycobacterium, are capable of biosynthesizing and storing TAGs over 20% of their own weight in lipids, a trait also known as oleaginicity.

Lignocellulosic biorefineries can achieve greater reductions in CO2 emission than petroleum-based biorefineries. It is anticipated that recent progress in lignocellulosic biorefinery technology will decrease production costs. High-value-added lipids, such as middle-chain fatty acids for use in health foods, are synthesized by oleaginous yeasts. Currently, such lipids are produced from animals and plants; they are expensive and not economically competitive. If lignocellulosic biomass or waste woody biomass could be used as feedstock, the cost of such lipids could be reduced, and new industries could develop.

U.S. Provisional Pat. Appl. 63/367,874 provides for an economical and efficient method to fractionate an enzyme inhibitor free cellulosic pulp from woody biomass that can further be converted into cellulosic sugar using standard enzymes. Two additional co-products of this process are novel hydrophobic liquid biohydrocarbon and a self-generated hydrophobic biosolvent blend providing for a very high yield of valued products from woody biomass. Thus, the sugar from this process can be used in various oleaginous yeasts to produce a lipid oil. The excess hydrophobic biosolvent can then be used within this disclosure for washing, extraction, and esterification of lipid based free fatty acids and the hydrophobic biohydrocarbon and be blended with the modified free fatty acids from the lipids. Overall, this provides for a high yield of fuel intermediaries from woody biomass and residual waste wood as a renewable carbon fuel.

### Microbial Lipid Makeup

Lipids accumulate in cell biomass majorly in the forms of triglycerides, free fatty acids (FFA), polar lipids, sterols, hydrocarbon and pigments. Oleaginous microorganisms are historically defined as organisms in which lipid content exceeds 20%, and are thus promising candidates for producing fatty acids as sustainable biofuel precursors.

Yeasts and fungi are also favorable oleaginous microorganisms, showing rapid growth rates on simple carbon sources such as glucose derived from corn, sugar cane, or cellulosic biomass. Oleaginous microorganisms contain a vast range of lipid classes, such as acylglycerides, phospholipids, glycolipids, lipoprotein, free fatty acids, sterols, hydrocarbons, and pigments. These lipid classes have different chemical and physical properties such as polarity, viscosity, solubility, and cellular location that define their availability during extraction.

Lipids accumulate in different locations in the cells and play important roles due to their specific cellular functions. Lipid bodies, consisting primarily of TAGs and sterol esters surrounded by a phospholipid monolayer rich in characteristic lipid body proteins, are present in the cytoplasm as a form of energy storage. The extraction process is important for extracting high yields of oils from the lysed microbes. The ability of the self-generated biosolvent within this disclosure provides for the ability to penetrate the lipid bodies and improve the washing/extraction yields. Secondly the self-generated biosolvent blend can be used for further processing of the extracted microbial oil or microbial-based free fatty acids into fatty acid butyl esters.

### Microbial Lipid Cell Disruption and Homogenation

TAGs and FFAs are usually considered as the favored precursors for biodiesel and hydrocarbon-based biofuels such as renewable diesel or renewable jet fuel, while the other side chains of polar lipids (sugars, proteins, phosphorous-containing molecules) may inactivate downstream processing catalysts.

The oil containing microbes or oleaginous yeast can be homogenized to disrupt the cellular structure as a pretreatment. A form of microbial pretreatment, such as cell disruption, is often necessary to remove or weaken the protective cell walls of microorganisms to make the intracellular lipids more accessible in extraction. Most studies in wet lipid extraction focus on cell disruption, which seemingly to be the sole factor influencing lipid recovery.

An ideal microbe disruption process cannot only assist lipid extraction by removing cell wall barriers, but should be able to increase mass transfer and simplify downstream processing as well.

The kinetics of lipid extraction from wet microbial slurry and lipid extraction efficiency was shown to typically increase with an increase in the speed of agitation, extraction temperature, pressure and the degree of cellular disruption. Various known methods can be used for cell disruption and homogenization used by those skilled in the art including, but not limited to high pressure homogenation, bead milling, and ultrasound, each of which are discussed below.

High-pressure homogenization (HPH) has been used to assist lipid extraction from various types of microbial mixture. HPH is currently used in industry for high-value protein recovery because it is simple to operate and scale up. High-pressure homogenization can also include the usage of supercritical processing with the addition of a hydrophobic blend of biosolvents that can penetrate the cellular wall structure of the microbe.

Cell disruption can be achieved by grinding biomass against the solid surfaces of beads during violent agitation. Bead milling has been applied to microalgae, bacteria, yeast, and fungal biomass to assist lipid extraction.

Ultrasound-induced cell disruption can improve lipid extraction from microalgae, yeast, and fungi. The disruption rate for ultrasonication follows a parabolic relationship with initial cell concentration, and lipid yields decreased dramatically when biomass concentration increased in the slurry.

### Fat Splitting Process

Oil splitting (or hydrolysis) can be performed with high-pressure steam, resulting in the formation of crude split of fatty acids and crude glycerin dissolved in the excess water. This process also is called the Colgate/Emery process that has been commonly used for decades in various industries.

In the splitting process, hydrolysis reaction proceeds in stages whereby the fatty acid radical in a triglyceride is displaced one at a time: from triglyceride to diglyceride, to monoglyceride and finally to glycerol:
TG+H2O <-->DG+FFA
DG+H2O <-->MG+FFA
MG+H2O <-->Glycerol+FFA

An incomplete fat splitting process will result in a product that contains a mixture of mono-, di- as well as up to some extent triglyceride. Fat splitting is a reversible reaction. When equilibrium is reached, the rate of hydrolysis will be equal to re-esterification. Thus, glycerin (by-product) must be drawn continuously for the completion of forward reaction.

### Example Process Description

In one embodiment, hydrolysis process typically occurs at temperature of 245˜255° C. and pressure between 55˜60 bar.
a) De-aeration

Crude oil is heated and de-aerated under vacuum to remove un-dissolved gasses before entering subsequent high temperature splitting section.
b) Splitting

In the Splitter, fatty acids are produced by counter-current splitting of oil with steam under temperatures up to 260° C. and pressures up to 60 bar.
c) Fatty Acid Drying

The fatty acids from splitter are flashed and dried to remove the residual moisture. The crude fatty acids are relatively dark in color and contain impurities.
d) Glycerin Water Pre-Concentration
   Crude glycerin is discharged from the bottom of splitter continuously. Crude glycerin water is first flashed before entering the evaporation stage.
   Concentration of crude glycerin obtained after the multiple effect evaporators is about 25%˜35%.
   Water flashed from crude glycerin is used for heat recovery in the multi-stage evaporators before pumped back to the splitter for reuse.

### Washing Extraction Process

After the conversion of the oil microbes to a free fatty acid from the fat splitting process, the FFA stream and any residual cell bodies that still contain FFA can be washed using a complex blend of a hydrophobic biosolvent. This disclosure utilizes a complex blend of hydrophobic biosolvents derived from a cellulosic refining process comprising butanol, butyl acetate, butyl esters, furans, wood extractive esters, and acids in which esters and furans are created within the process.

In evaluating various biosolvents for the washing extraction process, it is notable that butanol and higher carbon chain alcohols have a higher absorption rate into the microbial cells than other lower carbon chain alcohols such as methanol or ethanol. The use biosolvent that comprises butanol and other butanol derivatives results in a positive impact of intracellular microbial oil on absorption of the biosolvent, unconstrained by the yeast cell wall. Typically rapid absorption can be seen within a couple hours of contact of the microbes and biosolvent. This can facilitate subsequent intercellular microbial oil recovery.

In addition, the ability of the biosolvent blend to permeate the microbial cell was structure could further assist in downstream conversion of the cell bodies into various additional products and improve the microbial oil extraction yields. Permeability of the cell wall by the butanol and butanol derivatives within the biosolvent provides for means to weaken the cellular wall structure and improve microbial oil recovery, and assist in downstream processing.

Although embodiments first process the oil microbes through homogenization, fatty splitting, then a wash/extraction processing use a blend of hydrophobic biosolvents, in another embodiment the biosolvent can be mixed with the microbes at the beginning of this process given the ability of the butanol and butanol derivative to penetrate the cellular wall structure. In one embodiment, the biosolvent blend is mixed with the microbial admixture and processed under supercritical parameters that washing, extraction and optionally esterification can occur “in situ”.

Thus, the biosolvent can be added to the microbes, homogenized microbes, fat split microbes, free fatty acids or in combination. One embodiment is the addition and usage of the biosolvent after the FFA conversion process.

The biosolvent washing extraction process can be done at an elevated temperature wherein the biosolvent can form an emulsion with water at such elevated temperatures, but can phase separate upon cooling. The biosolvent blend of this disclosure is miscible in water only at elevated temperatures and upon cooling will phase separate thus allowing fractionation and recovery of an aqueous layer comprising glycerin, microbial cell bodies, and water as one layer and a hydrophobic layer comprising fatty acids, free fatty acids, or fatty acid esters based on based on order of addition within this process.
Washing Extraction Process
Temperature
Shear
Time

### Conversion of FFA to Fatty Acid Esters

In one embodiment, the free fatty acids are converted to fatty acid esters by means of esterification. Various methods can be used for the esterification of the FFA's including, but not limited to supercritical processing FFA's with an alcohol, acid or alkaline processing or other known methods for esterification.

In another embodiment, the addition of the biosolvent to free fatty acids can be esterified using supercritical methods given the biosolvent comprises a percentage of butanol, acetic acid and formic acid.

In another embodiment, the biosolvent can be added directly to the microbial broth at the beginning of the process and then can be esterified without the need for fat splitting or potentially homogenization given the ability of the butanol within the biosolvent to penetrate the microbial cellular wall structures.

In another embodiment, various acids can be used for esterification of the free fatty acids including, but not limited to, acetic acid, acetic anhydride, formic acid, oxalic acid, citric acid, methane sulfonic acid, or combinations thereof.

### Process of Supercritical Esterification of FFA's

The microbial free fatty acids can be esterified using the biosolvent into a fatty acid butyl ester that can be further blended with the hydrophobic biohydrocarbon either in liquid or solid form. Supercritical processing can be used in this process by blending the FFA with the hydrophobic biosolvent and process under supercritical conditions.

### Fatty Acid Butyl Ester

The disclosure provides for the use of a novel biosolvent derived from cellulosic fuel refining processes that is used for washing and extraction of microbial oil or free fatty acids from microbes. In addition, the same biosolvent that comprises a portion of butanol and butanol derivatives can be used for esterification of the free fatty acids or even the whole oil microbe.

Methanol is a widely used alcohol to produce biodiesel and ethanol to some extent. Other higher molecular weight alcohols like pentanol, propanol, and butanol are very rarely utilized for biodiesel synthesis. Among them butanol has some interesting advantages such has higher miscibility with lipid resources, higher contribution to initial mass transfer in reaction, higher boiling point of butanol permits conduction of reaction process at higher temperature to achieve faster rate of reaction.

The value of acid number or quantity of free FFA content of raw oil is one of the key parameters to decide the suitable transesterification process. Higher FFA content of raw oil (2.5% and above) requires pre-treatment prior to transesterification process. In the pre-treatment step, the esterification process can be carried out with acid catalyst to reduce the acid value to a required amount. If the value of FFA is not reduced to less than 2% in the first step, further esterification also can be repeated. Once the amount of FFA is reduced considerably, transesterification process can be carried with base catalyst.

The quality of the biodiesel is affected by the total count of carbon in the structure, amount of carbon double bond in the structure and quantity of unsaturated fatty acid of the resource oil. The relationship between physicochemical properties of biodiesel and chemical composition of the raw oil is important to select the desirable resource for good quality biodiesel.

The characteristics of flow of the biodiesel at low temperatures are improved when the raw oil contains low level of saturated fatty acids. Raw oil with low unsaturated fatty acid group will improve the oxidation stability and cetane number of the biodiesel. The viscosity and density are two controversial properties. The degree of saturation and chain length of the raw oil are directly proportional to the viscosity and inversely proportional to the density. At the outset raw oils with high mono unsaturated and low in both saturated and poly unsaturated fatty acid will produce good quality biodiesel.

Method of producing the fatty acid butyl ester from microbial oil and the biosolvent generated from cellulosic refining process can be processed in various methods. Supercritical esterification is included within this disclosure wherein as the free fatty acids are subjected to supercritical conditions with the biosolvent. Other standard means of creating the fatty acid butyl ester can be done using lower temperatures and pressure by the addition of acids and/or an alkaline material.

### Blending With Hydrophobic Biohydrocarbon

Although the present disclosure provides for a fatty acid ester using a blend of hydrophobic biosolvents for washing and extraction, the disclosure also provides for the blending of the fatty acid ester with a hydrophobic biohydrocarbon.

Liquid to Liquid—In one embodiment, the liquid microbial fatty acid ester can be blended with the liquid hydrophobic biohydrocarbon prior to the biosolvent recovery. The excess biosolvent provides for a homogenous liquid admixture. After blending, the admixture can be distilled to recovery the biosolvent.

Oxygen Content—The blend of hydrophobic biohydrocarbon and fatty acid ester will have a lower oxygen content than the hydrophobic biohydrocarbon alone. The further addition of a hydrogen donor to the liquid blend of biohydrocarbon, biosolvent and fatty acid esters is included within the disclosure to further reduce the oxygen content and provide additional advantages in downstream fuel processing.

Hydrogen Donor—Butanol can be a hydrogen donor in which the blending of the fatty acid ester that has been esterified with the biosolvent that comprises a significant percentage of butanol provides for the mechanism to increase hydrogen in the fuel intermediary.

Ratios of addition—The hydrophobic biohydrocarbon in its “organic layer” state comprises about 6-12% of the biohydrocarbon on a solids basis and the balance is the biosolvent blend. Ratios of free fatty acids or fatty acid esters can be blended from 1% to 99% biohydrocarbons, on a solids basis, to the free fatty acid or fatty acid ester.

Solvent Recovery Process—Upon blending the hydrophobic biohydrocarbon in its organic layer state and the free fatty acid or fatty acid ester the blend has extra biosolvent that can be removed by distillation. Distillation temperatures can control the specific biosolvent blend recovery makeup.

EFFECT OF FURFURAL within the BIOSOLVENT fatty acid butyl esterification.

The cellulosic refining process has the ability to create “excess” biosolvent each cycle based on the conversion of the hemicellulosic fraction of the woody biomass to various organic furans including furfural that make up a portion of the biosolvent blend. These organic furans are contained within the hydrophobic biosolvent blend.

The disclosure provides for the ability to recovery the biosolvent in which the furans are recovered in the solvent, or that the furans such as furfural can be left within the fatty acid butyl ester. Downstream hydrogenation processes can then convert the furfural fraction of the fatty acid butyl ester in to C4 and C5 mono-alcohols or processed into higher molecular carbon chain fuel intermediaries.

In another embodiment the furfural portion of the biosolvent can be converted back into butanol. A chemical reaction is shown with furfural combining with hydrogen over a catalyst to form C4 and C5 mono-alcohols such as 1-butanol, 1-pentanol, 2-pentanol and tetrahydrofurfuryl alcohol.

### Esterification of the Microbial Cell Structure

In another embodiment the residual cell bodies can be processed and used within the fuel intermediary or processed by means of esterification into additional products. In one embodiment of an oleaginous yeast, the cell wall of Trichosporon cutaneum consists of 11% protein, 63% neutral carbohydrate, 9% glucosamine and 13% glucuronic acid. The sugars include glucose (32%), mannose (6%) and traces of xylose and galactose.

In another embodiment, the residual cell bodies can be processed back into the digestion stage of the cellulosic refining process along with the woody biomass.

In another embodiment the cell bodies can be esterified using high amounts of a organic carboxylic acid such as citric acid, oxalic acid or formic acids blended with an alcohol.

### Corn Oil Option

The disclosure also provides for the usage of crude corn oil from corn ethanol production with microbial oil or microbial free fatty acids. The disclosure also provides for the usage of corn oil without blending with microbial oil. The disclosure also provides for the usage of crude soybean oil or corn oil that can be converted to a free fatty acid or esterified fatty butyl acid that can be further blended with the hydrophobic biohydrocarbon.

### Esterification of Oils Using Liquid Organic Phase Hydrophobic Biohydrocarbon

The disclosure also provides for the esterification of various oils using the liquid organic phase of the cellulosic refining process in which the organic phase includes the self-generated biosolvent blend and the hydrophobic biohydrocarbon materials. This biosolvent blend comprises many esters, butanol, butanol derivatives and acids based on the cellulosic refining process.

In one embodiment, various oils including, but not limited to soybean oil, corn oil, microbial oil, vegetable oils, waste cooking oils, crude tall oil, tall oil fatty acids, tall oil pitch of blends thereof can be blended with the liquid organic hydrophobic biohydrocarbon blend and esterified using standard methods of esterification.

In another embodiment, additional acids can be added including, but not limited to acetic acid, acetic anhydride, oxalic acid, formic acid and citric acid.

In another embodiment, the blend already comprising butanol, acids and the oils or free fatty acids can be processed using supercritical processes to esterify the oils or free fatty acids without the usage of additional acids.

The benefit of adding additional esterified fatty acid esters is that this blend will have reduced oxygen content that will provide for an advantage in downstream hydrocracking based on less hydrogen being required to produce a drop in fuel.

### Liquid Organic Phase Hydrophobic Biohydrocarbon Esterification

Within the woody biomass refining process, the hydrophobic biosolvent blend is added to ground woody biomass and water. The biosolvent blend comprises: butyl acetate, butyl ester, butanol, oleoresins, organic furans, fatty acids, rosins, terpenes, wood extracts, hemicellulose derivatives, such as furfural and 5-hydroxymethylfurfural (“HMF”), acetic acid, and formic acid. Other than butanol, the balance of the hydrophobic biosolvent is “self-generated” within the refining process, thus having the ability to create excess biosolvent. Given that the biosolvents comprises various acids and alcohols, the non-cellulose portion is subjected to esterification. The blend of hydrophobic biosolvents and the esterified non-cellulose fractions of wood represent the “hydrophobic liquid organic phase biohydrocarbon” material. If the solvent is recovered by various means of evaporation or distillation, the biosolvent can be recycled and the remaining hydrophobic biohydrocarbon is in a solid state with thermoplastic characteristics.

Thus, the liquid organic phase biohydrocarbon comprising both the dissolved solids and biosolvent are already esterified to a certain degree in situ. By esterification of the free fatty acids using the biosolvent, the esterified free fatty acids are then compatible with the liquid organic phase biohydrocarbon as to create a fuel intermediary liquid feedstock that can be further processed into gasoline, diesel or aviation fuels.

### Process 1

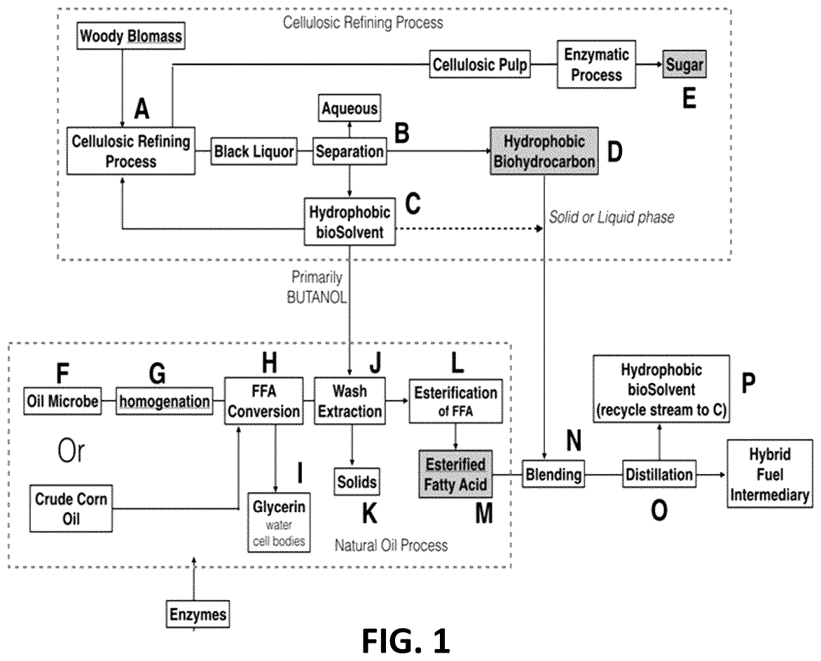

Referring now to Process 1 as shown in FIG. 1, in one embodiment the cellulosic refining process takes woody biomass and digests, fractionates and depolymerizes fraction of the wood using a hybrid hydrophobic biosolvent in a sealed reaction vessel under sufficient heat and pressure to depolymerize and convert a portion of lignin to hydrophobic phenols, convert the hemicellulose to various hydrophobic furans, and esterify wood extracts into hydrophobic esters. In addition, the process separates an enzyme “inhibitor reduced” cellulosic pulp for later enzymatic conversion to sugars (E) in which the first step after the primary heat/pressure conversion process is the removal of this novel pulp.

The process produces a “black liquor” that can then be separated by gravity phase separation (B), wherein an aqueous layer is separated and removed leaving a blend of hydrophobic biosolvent and a hydrophobic biohydrocarbon comprising hydrophobic phenols, hydrophobic furans, hydrophobic esters and acids. The hydrophobic biohydrocarbon (D) can be either in a liquid form with the hydrophobic biosolvents remaining or in a melt-flowable solid with thermoplastic melt flowability characteristics. The hydrophobic biosolvent (C) can be separated by evaporation.

Hydrophobic BioSolvent—The hydrophobic biosolvent of the following disclosure is produced using a hydrothermal cellulosic refining process wherein woody biomass is processed using a hydrophobic solvent blend that includes butanol, butyl acetate, butyl esters, furans, wood extractives and acids whereas the solvent blend includes a non-polar hydrophobic biosolvent blend. Within the cellulosic biorefining process, the biosolvent is used to depolymerize, fractionate and convert the native lignin into separate groups of depolymerized phenols. The hydrophobic phenols, in combination with the hydrophobic biosolvent, is referred to herein as an organic layer. The hydrophobic biosolvents can be recovered from the organic layer by evaporation leaving a solid melt flowable hydrophobic biohydrocarbon as explained in earlier referenced U.S. Provisional Pat. Appl. 63/367,874.

In the second part of this process, an oleaginous yeast oil microbe (F) is homogenized (G) in which various methods of homogenization include, but are not limited to: pressure, heat, high shear, (INSERT). After homogenization, the homogenized oil microbe can then be then converted into a free fatty acid in an FFA conversion process (H) wherein the FFA conversion process can be a fatty splitting process (Colgate/Remy Process), an enzymatic process or a combination of both.

The free fatty acid conversion step can be completed by subjecting to a microorganism stream of at least X % moisture to a pressure of 1000 psig and at a temperature of 200 to 250 C for fifteen minutes. In this process, the by-products include glycerin, water and microbial cell bodies that can be processed separately into other products or materials or reused within either the cellulosic refining process or sugar to microbial oil process.

The washing extraction process (J) utilizes the hydrophobic biosolvent (C) to mix, wash and separate out any additional solids (K) and recapture higher yields of FFA's.

The unique blend of the hydrophobic biosolvents (hydrophobic butyl esters, butanol, hydrophobic furans, hydrophobic esters, and acids) with the free fatty acids are then optionally esterified (L) using various methods of esterification as to create an esterified fatty acid (M).

Blending of the esterified fatty acids and hydrophobic biohydrocarbon liquid phase (N) can blend the two components over a wide range of ratios from 10:90 to 90:10 esterified fatty acids to hydrophobic biohydrocarbon. The blend can be heated and mixed while blending. The liquid admixture of the esterified fatty acids and hydrophobic biohydrocarbons can then be distilled to recover a significant portion of the biosolvent that can be recycled to either the washing step (J) or back into the cellulosic refining process.

The resulting material is a hybrid fuel intermediary comprising a blend of esterified fatty acids and hydrophobic biohydrocarbon with a lower oxygen content than the biohydrocarbon itself providing for various downstream fuel processing advantages including less hydrogen requirement for the conversion into renewable diesel, aviation fuels or other drop in liquid fuels.

Process 2—Option 2 Embodiment (in situ esterification)

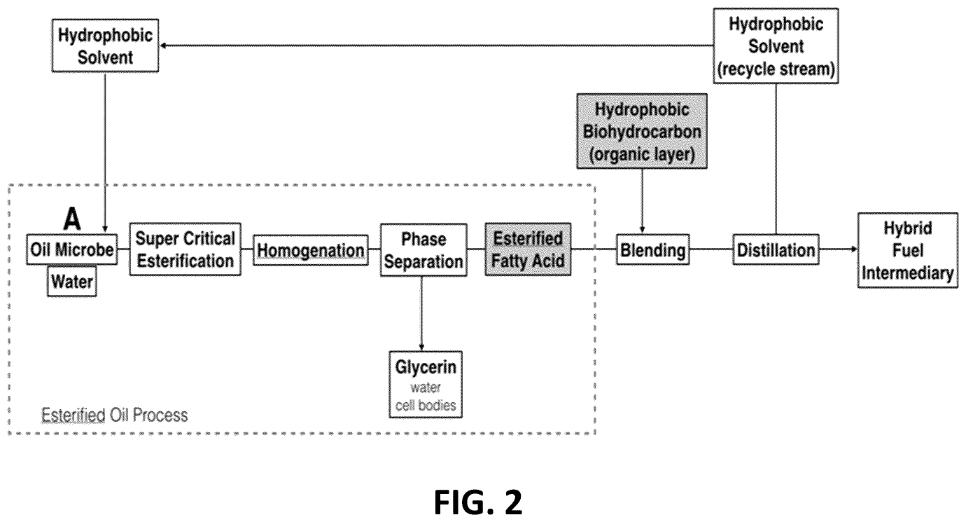

Referring now to PROCESS 2 as shown in FIG. 2, in another embodiment of the disclosure, the esterification of the fatty acid with the biosolvent can be done “in situ” with the oleaginous oil microbe itself. FIG. 2 illustrates a process wherein an oil-containing microbe (A) is first blended with the hydrophobic biosolvent (?) The ratios of microbes to hydrophobic biosolvent can range between 1 parts microbe to 100 parts biosolvent, or 1 part microbe to 25 parts biosolvent, or 1 part microbe to 10 parts biosolvent or 1 part microbe to 1 part biosolvent, or 1 part microbe to 0.2 parts biosolvent.

The admixture is then esterified using a supercritical esterification wherein the butanol portion of the biosolvent reacts with the oils under supercritical conditions to form an esterified free fatty acid. Butanol has the ability to absorb through the cellular walls of the microbe. Thus, by esterifying the oil microbe we can also positively affect the yields of FFA's from the microbe. After supercritical esterification, the material can be homogenized by means of heat, pressure or kinetic energy, which then proceeds to a phase separation process (?). The esterified fatty acids and hydrophobic biosolvent separate from the water, glycerin, and cell bodies by means of gravity phase separation.

At this point, the esterified fatty acids and hydrophobic biosolvent also comprising a portion of esters, are blended and distilled to recover and recycle a portion of the biosolvent into a hybrid fuel intermediary.

### EXAMPLES

### Experiment 1—Softwood vs. Hardwood of Biohydrocarbon Oxygen Content Based on Increased Free Fatty Acids

Hard wood and softwood (yellow pine) were ground into chips. The soft wood pine had higher levels of triacylglycerols, free fatty acids and phospholipids than the hardwood. Typically the most abundant fatty acid of the pine triaglyceride in the softwood is oleic, linoleum, palmitic, and linolenic acids. The wood chips were separately blended with a self-generated biosolvent from the cellulosic refining process and water. The admixture was placed into a PARR reactor and heated to 180 C for 1 hour. The materials were cooled and filtered to remove the cellulosic pulp. After sufficient cooling the liquid black liquor fraction phase separated into an aqueous and hydrophobic organic layer in which the organic layer was removed. The organic layer was then distilled as to recover the biosolvent blend leaving a solid hydrophobic biohydrocarbon. The two samples of biohydrocarbon (softwood and hardwood) were then tested for carbon, hydrogen and oxygen content.
Energy
Softwood Yellow Pine 12,314-13,000 BTU/lb
Hardwood Poplar 11,461-11,743 BTU/lb
Carbon
Softwood Yellow Pine 70.59%
Hardwood Poplar 62.5%
Oxygen
Softwood Yellow Pine 23.7%
Hardwood Poplar 30.75%
Hydrogen
Softwood Yellow Pine 5.37%
Hardwood Poplar 5.05%

The two materials were also evaluated by GC/MS and additional laboratory testing showing that the free fatty acid within the softwood esterified and was compatible with the biohydrocarbon producing a lower oxygen content. The softwood had higher levels of natural fatty acids from the wood extract or “wood resin” portion of the pine wood, thus this conversion of the fatty acids to an ester provides for a lower oxygen content that is advantageous in downstream hydrogenation processes to make a fuel.

Experiment 2—Compatibility of microbial oil to biosolvent (hot vs cold as to show the phase separation and compatibilities)

Experiment 3—Blending of a “ester” with the biosolvent and organic layer separately (ester maybe biodiesel and/or butyl fatty acid ester from our recovery solvent and oil)

Experiment 4—Oil microbes and recovery solvent under supercritical conditions (esterification and compatibility tests. phase separation afterward)

Experiment 5—Free fatty acid esterification using biosolvent—biosolvent with ffa, biosolvent with FFA and acid (approximately 6/1 ratio of butanol to ffa and additional acid) process at lower temperatures than supercritical

Experiment 6—Cell body esterification.

Experiment 7—glycerin esterification (HySoy g2 process)

Experiment 8—FIG. 1 process in whole.

Experiment 9—FIG. 2 process in whole.

Experiment 10—Esterification of various oils using organic layer hydrophobic. biocarbon/biosolvent blend.

### NON-LIMITING ILLUSTRATIVE EMBODIMENTS

The following non-limiting illustrative embodiments are non-limiting examples of embodiments disclosed in the subject application. However, these embodiments are to be understood to be presented for the purposes of illustration only and do not in any way limit the scope of the present disclosure described or contemplated herein.

Illustrative embodiment 1. A method comprising the steps of recovering free fatty acid from a lipid producing microorganism by subjecting the microorganism to a free fatty acid conversion step followed by a hybrid biosolvent washing/extraction process.

Illustrative embodiment 2. The method of any of the preceding Illustrative embodiments, wherein the microorganism comprises at least one of a type of yeasts, fungi, bacteria and microalgae.

Illustrative embodiment 3. The method of any of the preceding Illustrative embodiments, wherein the microorganism comprises an oleaginous yeast.

Illustrative embodiment 4. The method of any of the preceding Illustrative embodiments, wherein the microorganism is first concentrated from the broth or liquid stream through filtration.

Illustrative embodiment 5. The method of any of the preceding Illustrative embodiments, wherein the microorganism is subjected to a step that ruptures most or all the cell walls by a homogenization process.

Illustrative embodiment 6. The method of any of the preceding Illustrative embodiments, wherein the homogenization process can include at least one of heat, pressure, high shear, supercritical, pressure differential, or combinations thereof.

Illustrative embodiment 7. The method of any of the preceding Illustrative embodiments, wherein the whole or ruptured microorganism is subjected to a free fatty acid conversion step.

Illustrative embodiment 8. The method of any of the preceding Illustrative embodiments, wherein the free fatty acid conversion step comprises enzymatic conversion.

Illustrative embodiment 9. The method of any of the preceding Illustrative embodiments, wherein the free fatty acid conversion step is completed by subjecting to a microorganism stream of at least X % moisture to a pressure of 1000 psig and at a temperature of 200 to 250° C. for fifteen minutes.

Illustrative embodiment 10. The method of any of the preceding Illustrative embodiments, wherein the free fatty acid conversion step comprises a fat splitting (Colgate/Emery) process.

Illustrative embodiment 11. The method of any of the preceding Illustrative embodiments, wherein the converted or partially converted microorganism is subjected to a separation step to remove solids from the liquid stream.

Illustrative embodiment 12. The method of any of the preceding Illustrative embodiments, wherein the separation step comprises at least one of centrifugation, filtration distillation, or combination thereof.

Illustrative embodiment 13. The method of any of the preceding Illustrative embodiments, wherein the separated solids contain suspended solids, lipids, and/or free fatty acid.

Illustrative embodiment 14. The method of any of the preceding Illustrative embodiments, wherein the biosolvent comprises at least one of: butyl acetate, butyl ester, butanol, oleoresins, organic furans, fatty acids, rosins, terpenes, wood extracts, hemicellulose derivatives, such as furfural and 5-hydroxymethylfurfural (“HMF”), acetic acid, and formic acid.

Illustrative embodiment 15. The method of any of the preceding Illustrative embodiments, wherein the separated solids are subjected to a biosolvent blend washing step to recover a portion of any lipids or free fatty acids that remain with the solids.

Illustrative embodiment 16. The method of any of the preceding Illustrative embodiments, wherein the biosolvent washing step subjects the microorganisms and corresponding derivatives (Lipids, FFA, and remaining cell bodies) leaving the free fatty acid conversion step to a self-generated biosolvent to wash remaining solids (cell bodies).

Illustrative embodiment 17. The method of any of the preceding Illustrative embodiments, wherein the self-generated biosolvents are produced using a cellulosic refining process of woody biomass.

Illustrative embodiment 18. The method of any of the preceding Illustrative embodiments, wherein the biosolvent washing step produces a solvent stream comprising free fatty acid, fatty acid butyl esters, or lipids, derived from microorganisms and self-generated biosolvent.

Illustrative embodiment 19. The method of any of the preceding Illustrative embodiments, wherein the free fatty acid containing organosolv solvent stream is subjected to a separation process to separate or partially separate free fatty acids from the solvent (evaporation, distillation, other).

Illustrative embodiment 20. The method of any of the preceding Illustrative embodiments, wherein the separated solids are subjected to an additional solvent removal step.

Illustrative embodiment 21. The method of any of the preceding Illustrative embodiments, wherein washed solids are used as feed, food, or esterified solids.

Illustrative embodiment 22. The method of any of the preceding Illustrative embodiments, wherein washed solids are esterified using the self-generated biosolvent blend under heat and pressure.

Illustrative embodiment 23. The method of any of the preceding Illustrative embodiments, wherein the converted or partially converted microorganism also includes a portion of free fatty acid generated from the cell bodies.

Illustrative embodiment 24. The method of any of the preceding Illustrative embodiments, wherein the self-generated biosolvent can further comprise water, ethanol, methanol, isobutanol or an additional alcohol.

Illustrative embodiment 25. The method of any of the preceding Illustrative embodiments, wherein the self-generated biosolvent can include depolymerized lignin or methoxyl phenols.

Illustrative embodiment 26. The method of any of the preceding Illustrative embodiments, wherein the free fatty acid generated from a microorganism is at least partially contained in a stream that contains a self-generated biosolvent and a liquid hydrophobic biohydrocarbon co-product from a cellulosic fuel process.

Illustrative embodiment 27. The method of any of the preceding Illustrative embodiments, wherein the hydrophobic biohydrocarbon comprises.

Illustrative embodiment 28. The method of any of the preceding Illustrative embodiments, wherein the stream that contains a free fatty acid, fatty acid butyl ester, self-generated bio solvent and liquid hydrophobic biohydrocarbon is subjected to an evaporation step to recover the self-generated biosolvent.

Illustrative embodiment 29. The method of any of the preceding Illustrative embodiments, wherein the stream is subjected to a solid removal step prior to, during and/or after the evaporation step.

Illustrative embodiment 30. The method of any of the preceding Illustrative embodiments, wherein the water content is less than 1%, 0.5%, or 0.2%.

Illustrative embodiment 31. The method of any of the preceding Illustrative embodiments, wherein the admixture of free fatty acid, fatty acid butyl ester, self-generated biosolvents and liquid hydrophobic biohydrocarbon are further esterified using at least one the group consisting of acid, carrier fluid, organic solvent, petrochemical solvent, and blends thereof.

Illustrative embodiment 32. The method of any of the preceding Illustrative embodiments, wherein the acid comprises an acid selected from the group consisting of acetic acid, acetic anhydride, oxalic acid, citric acid, formic acid, meth sulfonic acid, and blends thereof.

Illustrative embodiment 33. A fuel intermediary product comprising: a liquid hydrophobic biohydrocarbon; and a free fatty acid or fatty acid butyl ester.

Illustrative embodiment 34. The product of any of the preceding Illustrative embodiments, wherein at least a portion of the free fatty acid or esterified free fatty butyl acid is produced from a microorganism.

Illustrative embodiment 35. The product of any of the preceding Illustrative embodiments, wherein the microorganism comprises oleaginous yeast.

Illustrative embodiment 36. The product of any of the preceding Illustrative embodiments, wherein the microbial FFA is at least partially esterified using a biosolvent blend from cellulosic refining.

Illustrative embodiment 37. The product of any of the preceding Illustrative embodiments, wherein biosolvent blend is used to esterify the free fatty acids and hydrophobic biohydrocarbon.

Illustrative embodiment 38. The product of any of the preceding Illustrative embodiments, wherein the biosolvent comprises at least one of the group consisting of butyl esters, butyl acetate, hydrophobic organic furans, hydrophobic wood extracts, and combinations thereof.

Illustrative embodiment 39. The product of any of the preceding Illustrative embodiments, wherein the biohydrocarbon comprises at least one of the group consisting of hydrophobic phenols, hydrophobic furans, hydrophobic wood esters, hydrophobic butanol and butanol derivatives.

Illustrative embodiment 40. The product of any of the preceding Illustrative embodiments, wherein the hydrophobic biohydrocarbon and fatty acid butyl ester are at least partially esterified further using a carboxylic acid and/or carboxylic acid hydrogen donor.

Illustrative embodiment 41. The product of any of the preceding Illustrative embodiments, wherein the carboxylic acid comprises an acid selected from the group consisting of acetic, citric, formic, oxalic, and blends thereof.

Illustrative embodiment 42. A method of making an esterified free fatty acid fuel intermediary comprising: mixing a hydrophobic biosolvent with oil microbes; causing the Hydrophobic biosolvent to penetrate a cell structure of the microbes; processing the hydrophobic biosolvent and microbes under supercritical heat and pressure to form fatty acid butyl esters; homogenating the microbial cells; phase separating the esterified fatty acids and hydrophobic solvent from an aqueous phase; and evaporation recovery of a portion of the hydrophobic biosolvent.

1. A method of making an esterified free fatty acid fuel intermediary comprising:
a. mixing a hydrophobic biosolvent with oil microbes;
b. causing the hydrophobic biosolvent to penetrate a cell structure of the microbes;
c. processing the hydrophobic biosolvent and microbes under supercritical heat and pressure to form fatty acid butyl esters;
d. homogenating the microbial cells;
e. phase separating the esterified fatty acids and hydrophobic solvent from an aqueous phase; and
f. evaporation recovery of a portion of the hydrophobic biosolvent.

2. A method of recovering free fatty acid from a lipid producing microorganism, comprising the steps of:
subjecting the microorganism to a free fatty acid conversion step followed by a hybrid biosolvent washing/extraction process.

3. The method of claim 2, wherein the microorganism is selected from the group consisting of yeast, fungi, bacteria, and microalgae.

4. The method of claim 3, wherein the microorganism comprises an oleaginous yeast.

5. The method of claim 2, further comprising at least one step selected from:
concentrating the microorganism from a broth or liquid stream through filtration prior to the free fatty acid conversion step;
subjecting the microorganism to a homogenization process that ruptures at least a portion of cell walls of the microorganism; and
subjecting the converted or partially converted microorganism to a separation step to remove solids from the liquid stream, wherein the separation step comprises at least one of centrifugation, filtration distillation, or combination thereof.

6. The method of claim 2, wherein the free fatty acid conversion step comprises enzymatic conversion.

7. The method of claim 2, wherein the free fatty acid conversion step is completed by subjecting a microorganism stream to a pressure of 1000 psig and at a temperature of 200 to 250° C. for fifteen minutes.

8. The method of claim 2, wherein the free fatty acid conversion step comprises a fat splitting (Colgate/Emery) process.

9. The method of claim 2, wherein the biosolvent is selected from the group consisting of butyl acetate, butyl ester, butanol, oleoresins, organic furans, fatty acids, rosins, terpenes, wood extracts, hemicellulose derivatives, such as furfural and 5-hydroxymethylfurfural (“HMF”), acetic acid, formic acid, and combinations thereof.

10. The method of claim 2, wherein the biosolvent washing step produces a solvent stream comprising free fatty acid, fatty acid butyl esters, or lipids, derived from microorganisms and self-generated biosolvent.

11. The method of claim 2, wherein the free fatty acid containing organosolv solvent stream is subjected to a separation process to separate or partially separate free fatty acids from the solvent (evaporation, distillation, other).

12. The method of claim 2, wherein the free fatty acid generated from the microorganism is at least partially contained in a stream that contains a self-generated biosolvent and a liquid hydrophobic biohydrocarbon co-product from a cellulosic fuel process.

13. The method of claim 12, wherein the stream that contains a free fatty acid, fatty acid butyl ester, self-generated bio solvent and liquid hydrophobic biohydrocarbon is subjected to an evaporation step to recover the self-generated biosolvent.

14. The method of claim 2, wherein the water content is less than 1%.

15. A fuel intermediary product comprising:
a liquid hydrophobic biohydrocarbon; and
a free fatty acid (FFA) or fatty acid butyl ester.

16. The product of claim 15, wherein at least a portion of the free fatty acid or esterified free fatty butyl acid is produced from a microorganism.

17. The product of claim 16, wherein the microorganism comprises oleaginous yeast.

18. The product of claim 16, wherein the microbial FFA is at least partially esterified using a biosolvent blend from cellulosic refining.

19. The product of claim 15, wherein the biohydrocarbon comprises at least one of the group consisting of hydrophobic phenols, hydrophobic furans, hydrophobic wood esters, hydrophobic butanol and butanol derivatives.

20. The product of claim 15, wherein the hydrophobic biohydrocarbon and fatty acid butyl ester are at least partially esterified further using a carboxylic acid and/or carboxylic acid hydrogen donor.
